(19) United States
(12) Patent Application Publication
(10) Pub. No.: US 2008/0008769 A1
(43) Pub. Date: Jan. 10, 2008

(54) Treatment of urinary tract infections

(76) Inventors: Gregg Siegel, San Antonio, TX (US); Phyllis Siegel, San Antonio, TX (US)

Correspondence Address: JOHN LEZDEY & ASSOCIATES, Suite 118, 2401 West Bay Drive, Largo, FL, 33770, US

(21) Appl. No.: 11/481,210

(22) Filed: Jul. 5, 2006

(51) Int. Cl. A61K 33/40; A61K 33/36; A61K 31/19

(52) U.S. Cl. 424/669; 424/670; 424/671; 424/613; 514/557

(57) ABSTRACT

The invention provides a method for treating urinary tract infections in a patient by providing a mouth rinsing composition which releases free molecular iodine into the mucous membranes which enters into the urinary tract.

### FIELD OF THE INVENTION

There is provided a method for treating urinary tract infections systemically by administering a composition forming free molecular iodine in an oral rinsing solution. More particularly, there is provided a method for treating bacterial infections of the urinary tract by a mouth rinsing solution.

### BACKGROUND OF THE INVENTION

Bacterial infections of the lower urinary tract are very common. Infections usually occur by the ascending route (from perineum to vagina to urethra to bladder). Escherichia coli is the most common bacterium and accounts for about 75% of community-aquired infections. Klebsiella sp. together account for 10 to 15% of the remainder. Coagulase-negative streptococeus faecalis and staphylocci each cause about 2 to 3%. Patients who are subject to instrumentation catheters (catheterization) are likely to be infected with Serratia maisessens, pseudomonas aeruginosa, S. epidermidis and Candida sp. Typical treatment is with an antibiotic such as amoxicillin, cephalosporins and cotrimoxazole.

Yeast and fungal infections are found in the lower urinary tract of girls and women which are commonly treated with antibiotics. Amphotericin B and flucytosine are preferred for use in the treatment.

Because of side effects, bacterial resistance and the association of antibiotics and asthma, it has become desirable to use non-antibiotic treatment for adolescents, pregnant women and mild cases of urinary tract infections.

Although mouthwashes are standard in oral hygiene, they have generally been used to mask halitosis. Several mouthwashes that have been marketed for the reduction of bacteria and the prevention of plaque build up and gingivitis generally rely on a combination of alcohols (e.g. thymol, eucalyptol, ethanol; such as Listerine®), a combination of alcohols and quaternary amine (e.g. ethanol, cetylpyridinium chloride; such as Scope®) or other oral surfactants (see U.S. Pat. No. 4,657,758), or of alcohol and chlorhexidine digluconate (Peridex® from Proctor and Gamble). However, the use of high amounts of alcohol in formulations tend to produce unpleasant side effects including pain and stinging of the oral mucosa, foul aftertaste and discoloration of teeth. Use of chlorohexidine results in stained teeth. Prior art attempts to address this issue have included the use of a cetylpyridinium chloride in the presence of an oral surfactant (lander Alcohol Free Mouthwash from the Lander Company, Inc.) and the use of stabilized chlorine (RetarDent® from Rowpar).

None of the prior art reference provide a means for providing molecular free iodine to be used systemically.

U.S. Pat. No. 3,215,627 discloses a method for use in the disinfection of swimming pools. A pH range of 7 to 8 is taught as critical to both U.S. Pat. Nos. 3,232,869 and 3,215,627. The range of free molecular iodine that is generated according to the method of the '627 patent is between 0.2 and 0.4 ppm. This patent also teaches that an iodide salt is of no value because iodine release is erratic and unpredictable and because it is not possible to achieve or maintain a desired iodine level.

U.S. Pat. Nos. 3,215,627 and 3,232,869 identify a concentration range of 0.1 to 1.0 ppm of iodide ion as the practical concentration range. This concentration of iodide equates to a theoretical maximum free molecular iodide concentration of 0.85 ppm. Moreover, both the '627 and '869 patents teach that a pH in the range of 7 to 8 is critical.

### SUMMARY OF THE INVENTION

In accordance with the invention there is provided a method of treating urinary tract infections by the oral rinsing of the mouth with an aqueous composition comprising an effective amount of a monobasic iodide salt to provide at least 30 ppm of available iodine, an effective amount of an organic acid having up to eight carbon atoms, an effective amount of at least one oxidizing agent to yield free molecular iodine and a buffer, preferably a phosphate buffer, which can be incorporated in mouth washing preparations. More particularly, the present composition comprises a monobasic iodide salt which is an alkali metal salt, preferably sodium, calcium, magnesium or potassium iodide in an amount of at least about 0.01 to 0.5% by weight, more preferably about 0.01 to 0.1%, an organic acid having up to eight carbon atoms, preferably selected from the group consisting of citric acid, ascorbic acid, and oxalic acid or the salts thereof in an amount of about 0.1 to 1% by weight, preferably 0.1 to 0.5%, an oxidizing agent, a buffer and an aqueous solvent.

The oxidizing agent is preferably the alkali metal salt of a per acid or urea hydrogen peroxide which is present in an amount of at least about 0.01 to 1.0% by weight. There is an available iodine of at least 30 ppm, preferably about 80 to 300 ppm.

The composition is buffered to a pH of 2.3 to 6.0, preferably 3.0 to 3.5.

It is a general object of the invention to provide a non-staining formulation for use in rinsing the mouth to provide a release of free molecular iodine into the urinary tract.

It is a further object of the invention to provide a composition which treats urinary tract infections.

It is a further object of the invention to treat vaginal yeast infections.

It is yet another object to provide a composition which can treat bacterial infections of the urinary tract.

It is another object of the invention to provide a method for treating fungal infections of the urinary tract.

It is still another object of the invention to treat thrush.

### DESCRIPTION OF THE PREFERRED EMBODIMENTS

In accordance with the present invention there is provided an aqueous antimicrobial composition that can be used alone or incorporated with an antibiotic which comprises:
A. an effective amount of a monobasic iodide salt to provide at least 30 ppm of available iodine to said composition, preferably about 80 to 300 ppm;
B. an organic acid having up to eight carbon atoms;
C. an oxidizing agent, preferably an alkali salt of a per acid or urea hydrogen peroxide;
D. a buffer which can include the organic acid of part B, and;
E. an aqueous solvent, said composition having an available iodine content of at least 30 ppm, preferably about 80 to 300 ppm.

The preferred composition which may be added to an existing or prepared preparation comprises;
1) at least about 0.01 to 5% by weight of a monobasic iodide salt which is an alkali metal salt, preferably sodium or potassium iodide, so as to supply at least 80 to 300 ppm of available iodine;
2) about 0.01 to 2.0% by weight preferably about 0.01 to 2.0% by weight of an organic acid having up to eight carbon atoms, more particularly selected from the group consisting of citric acid, ascorbic acid and oxalic acid, or the alkali salts thereof,
3) about 0.009 to 1% by weight of an oxidizing agent selected from the group consisting of alkali salts of peroxide, sodium percarbonate, sodium perborate, sodium persulfate, urea hydrogen peroxide, peroxidase, ascorbic acid and mixtures thereof or other oxidizing agents used in the whitening of teeth having an oxidation potential greater than −0.54 electron volts, and
4) water, the composition can be buffered to a required pH to be used alone or alternatively after incorporated into a mouthwash preparation.

The composition is effective against a wide variety of various aerobic, anaerobic and facultative species, including Candida albicans, S. aureas, T. denticola, P. intermedia, cectinomyces, viscosus, P. gingivalis, S. sangrias, S. mutans, A. viscosus and A. naeslundii.

In general, the acid necessary to supply the required pH to the overall composition can be any organic or inorganic acid which does not chemically react with the other components, such a hydrochloric acid, phosphate salts, phosphoric acid, sulfuric acid, citric acid, acetic acid, preferably the organic acids or phosphate salts such as calcium pyrophosphate. The operating pH range for the composition is 2.3 to 4.0 and preferably, from about 2.8 to 3.3. The pH of an aqueous solution comprising the above enumerated components of the invention is determined by employing an aqueous solution of 0.5%, by weight, total of active components typically at a glass electrode, to precisely define the acidity of the composition.

In practice, the amounts of each of the components of the overall composition can range widely from 0.009 parts to 40.0 parts by weight depending upon use. The balance after allowing for the acid is usually a physiologically acceptable solvent, such as water or a lower (C1-C4) monohydric aliphatic alcohol, for a total of 100 parts or more. Where water is employed, small amounts of a lower alkyl alcohol, such as ethanol or propanol, may be added thereto to provide easy formulation. The pH of the total composition is then adjusted to the requisite pH by adding a suitable inorganic or organic acid thereto. The critical percent is to maintain an available iodine content of at least 30 ppm, preferably 80 to 300 ppm. The lower pH has the greater amount of iodine parts per million.

The composition is buffered to a pH of 2.2 to 6.0, preferably about 3.0 to 3.6. There is an available iodine of at least 30 pm, preferably about 80 to 300 ppm.

The solvent can comprise at least 50% water with an alkanol having 1 to 4 carbon atoms or can be 100% water, preferably the alcohol is ethanol and/or isopropanol.

Optional ingredients can be a magnesium salt, namely magnesium iodide or magnesium sulfate. The magnesium iodide can be used to provide the source of molecular iodine alone or in combination with sodium or potassium iodide. The magnesium salts are also anti-microbial.

In general, the required pH of the overall composition can be any organic or inorganic acid which does not chemically react with the other components, such as hydrochloric acid, phosphate salts, phosphoric acid, sulfuric acid, citric acid, acetic acid, preferably the organic acids and/or phosphate salts are utilized.

The lower pH has the greatest amount of iodine in parts per million.

The buffering agents may be utilized to maintain pH within the desired range of 2.3 to 6.0, or within the more preferred range of 3.0 to 3.5. Suitable buffering agents for inclusion in the compositions of the invention include potassium phosphate, mono or dibasic, glycine-glycine-HCl, potassium hydrogen phthalate-phthalic acid, citric acid-Na₂HPO₄, citric acid-KH₂PO₄—H₃BO₃-diethylbarbituric acid-NaOH, citric acid-sodium citrate, dimethylglutaric acid-sodium dimethylglutarate, acetic acid-sodium acetate, succinic acid-sodium succinate, potassium hydrogen phthalate-dipotassium phthalate, sodium cacodylate-cacodylic acid, sodium hydrogen maleate-disodium maleate, Na₂HPO₄—NaH₂PO₄, sodium bicarbonate-5% CO₂, imidazole-imidazole HCl, boric acid, sodium borate, and the following buffers known to one skilled in the art: Tris, MES, BIS-TRIS, ADA, ACES and PIPES. Enough buffer is added to maintain the pH below 6.0. In general, a buffer concentration of at least 5 millimolar is utilized.

Aqueous mediums suitable for use in the present invention include water, mixtures of water and alcohols (such as ethanol, and isopropanol), or mixtures of water and other water-miscible solvents. In general, an aqueous medium will be capable of dissolving iodide salts and will not react rapidly with free molecular iodine. In preferred embodiments, the aqueous medium is substantially non-toxic. In preferred embodiments, the aqueous medium is at least 50% water by volume so as to be synergistic with the alcohol.

According to the present invention there is provided a means of treating urinary tract infections by pathogens, yeast or fungus by rinsing the mouth with a composition of the invention so that free molecular iodine produced by the composition is absorbed by the mucous membrane and fed into the urinary tract. The number of rinses and the number of application will depend upon the type of infection, the severity of infection and the age of the patient as determined by the physician.

The formulations can be used to prevent the infections by washing the urinary tract when an infection is suspected. There are indications that the composition works systemically.

The compositions of the inventions can be used in combination with the conventional antibiotics in treating the various infections by aerobic and anaerobic pathogens such as Candida albicans, S. aureas, S. mutans, E-coli, Staphylococcus epidermis, Chlamydia trachomatis and the like.

The compositions are especially useful in cases which have become antibiotic resistant.

The following examples illustrate the invention:

### EXAMPLE 1

A mouth rinsing solution having 175 ppm of free molecular iodine and a pH of 2.2-3.6 is prepared by admixing the following ingredients:

| Ingredient | Wt. % |
|---|---|
| Sodium iodide | 0.056 |
| Potassium phosphate | 0.23 |
| Monohydrate | |
| Citric acid, anhydrous | 0.36 |
| Sodium perborate | 0.009 |
| Monohydrate | |
| Deionized water | q.s. |
| | 100% |

### EXAMPLE 2

Object: To study the effect of the iodine formulation of Example 1 at three different pH levels on metabolic activity of cells in Candida albicans.

Methods: The three iodine formulations used in this experiment were: 1) Iodine pH 5.0, 80 ppm Iodine; 2) Iodine pH 6.4, 80 ppm Iodine; and 3) Iodine pH 3.30, 150 ppm Iodine.

Candida albicans biofilm was grown in three 96 wells micro-titer plates for 24 hours. The wells were carefully emptied and washed three times with phosphate-buffered saline to remove unattached cells. In each plate, one row of eight wells was used as control. In other six rows of eight wells, the biofilm was exposed to 15 or 30 μL of each of the three formulations listed above for one, five and twenty minutes. After the exposure time, fluids from the wells were carefully aspirated and the biofilms were washed repeatedly with 100, 50 and 50 μL of PBS. A semi-quantitative measure of biofilm formulation was determined by using the XTT reduction assay of Ramage, G. et al (2001) standardized method for in vitro antifungal susceptibility testing of Candida albicans biofilm, antimicrobial agents and chemotherapy. 9:2475-2479. The percent inhibition of Candida albicans biofilm by these iodine formulations was compared to the biofilm in the control wells.

Results: The following table represents the summary of the percent inhibition of C. albicans biofilm formulation by the three formulations at one-half or full strength and for the three exposure times.

| | Percent Inhibition | | |
|---|---|---|---|
| | Exposure Time: | | |
| | 1 | 5 | 20 |
| | minute | minutes | minutes |
| Cells + Iodine pH 5.0 - 30 μL | 48.00 | 63.89 | 66.96 |
| Cells + Iodine pH 6.4 - 30 μL | 78.57 | 93.33 | 95.21 |
| Cells + Iodine pH 3.3 - 30 μL | 84.44 | 95.74 | 94.84 |
| Cells + Iodine pH 5.0 - 15 μL | 38.89 | 29.47 | 50.28 |
| Cells + Iodine pH 6.4 - 15 μL | 50.44 | 54.77 | 64.77 |
| Cells + Iodine pH 3.3 - 15 μL | 58.22 | 53.12 | 50.42 |

The percent inhibition by the Iodine formulation pH 3.3 at full strength was 84.44, 95.74 and 94.84 after 1, 5, and 20 minute exposure times, respectively, and that by the full strength iodine formulation at pH 6.4 was 78.57, 93.33 and 95.21 at the same exposure times respectively. The percent inhibition was similar at 5 and 20 minute exposures by both of these formulations. The percent inhibition by these two iodine formulations at one-half strength was between 29 to 65% at all three exposure times. The percent inhibition of C. albicans biofilm formation by the iodine formulation at pH 5.0 was 48.00, 63.89 and 66.96 after 1, 5, and 20 minute exposure at full strength and 38.89, 29.47 and 50.28 at one-half its strength.

Conclusions: The iodine formulation pH 3.3 was found to be more effective in inhibiting Candida albicans biofilm formation starting at a 1 minute exposure time.

### EXAMPLE 3

Purpose: To estimate the Iodine in Oral Rinse formulation before and after swishing in mouth for 30 seconds.

Method: Seven volunteers were given 15 ml each of Oral Rinse of the formulation of Example 1 which contained 140 ppm iodine, pH 2.85, which they swished around in their mouth for 30 seconds. The used formulation was collected from each volunteer in a separate container. Each used formulation was titrated to estimate iodine as per S.O.P. 100.006 and its pH was also measured. One volunteer happened to gargle the formulation.

Results: (Attach Data to Support): The average titratable iodine in the used Oral Rinse was 34.5 ppm (range 12.7 to 57.2 ppm) and the average pH was 3.00 (range 2.87 to 3.10). An average of 24.6% (Range 9.1 to 40.1%) iodine was present after the use. Thus the titratable iodine was reduced by an average of 105.5 ppm (Range 82.8 to 127.3 ppm) in the used formulation. The average percent reduction in titratable iodine was 75.4% (Range 59.1 to 90.9%). The loss of iodine was the largest in the used formulation obtained from the volunteer who gargled with it. The change in pH was not appreciable.

Conclusions: There is considerable reduction in titratable iodine after using the Oral Rinse formulation for 30 seconds. The pH was not changed appreciably. The composition can be used for treating urinary tract infections.

### EXAMPLE 4

An oral rinse was prepared by mixing the following ingredients:

| Ingredient | Wt. % |
|---|---|
| Citric Acid | 0.158 |
| Sodium Iodide | 0.0245 |
| Sodium Perborate | 0.033 |
| Potassium Phosphate Monobasic | 0.1 |
| Water | q.s. |

If desired, 0.1% Menthol and 0.03% Sucralose can be added in the above formulation and the quantity of water is adjusted accordingly.

The composition can be used to treat thrush.

### EXAMPLE 5

An oral rinse is prepared by admixing the following ingredients:

| Ingredient | Wt. % |
|---|---|
| Sodium Iodide | 0.0245 |
| Citric Acid | 0.157 |
| Sodium Persulfate | 0.0794 |
| Sodium Percarbonate | 0.033 |
| Water | q.s. |

### EXAMPLE 6

An oral rinse is prepared by admixing the following ingredients

| Ingredient | Wt. | |
|---|---|---|
| Potassium Mono or Dihydrogen | 0–1.0 | g |
| Phosphate | | |
| Sodium Iodide | 0.01–0.06 | g |
| Citric Acid | 0.01–0.5 | g |
| Sodium Perborate | 0.009–0.4 | g |
| Menthol | 0.1% | by weight |
| Sucralose | 0.03% | by weight |

| | |
|---|---|
| 1% usp water | q.s. |

The pH is adjusted to 3.3–3.8.

What is claimed is:

1. A method for treating urinary tract infections of a patient by administering into the mouth of a patient an oral rinsing composition comprising:
a) an effective amount of a monobasic salt to provide at least 30 ppm of available iodine.
b) about 0.1 to 1% of an effective amount by weight of an organic acid having up to eight carbon atoms;
c) An effective amount of an oxidizing agent to yield free molecular iodine;
d) An effective amount of a buffer to provide a pH of 2.3 to 6.0; and the remainder
e) an aqueous solvent,
said composition allowing free molecular iodine to be absorbed into the mucous membranes so as to enter the urinary tract.

2. The method of claim 1 wherein said monobasic salt is selected from the group consisting of sodium iodide, potassium iodide, magnesium iodide and calcium iodide.

3. The method of claim 1 wherein said solvent is water.

4. The method of claim 1 wherein the pH of the composition is about 2.5 to 3.5.

5. The method of claim 1 wherein the infection is bacterial.

6. The method of claim 1 wherein the infection is fungal.

7. The method of claim 1 wherein the infection is a vaginal yeast infection.

8. The method of claim 1 wherein said oxidizing agent comprises sodium perborate.

9. The method of claim 1 wherein said composition comprises:
a) about 0.1 to 5% by weight of sodium iodide;
b) about 0.01 to 2% by weight of citric acid;
c) about 0.009 to 1% of sodium perborate;
d) An effective amount of a phosphate buffer to provide a pH of 2.3 to 6, and the remainder
e) water or alcohol/water,
whereby there is provided at least 80 to 300 ppm of free molecular iodine.

10. (canceled)

11. The method of claim 9 wherein the patient has a fungal infection.

12. The method of claim 9 wherein the patient has a yeast infection.

13. The method of claim 9 wherein the patient is suffering from thrush.
